(19) United States
(12) Patent Application Publication
(10) Pub. No.: US 2015/0051576 A1
(43) Pub. Date: Feb. 19, 2015

(54) NESTED CANNULA TIPS

(71) Applicant: KONINKLIJKE PHILIPS N.V., EINDHOVEN (NL)

(72) Inventors: Karen Irene Trovato, Putnam Valley, NY (US); Willem Albert Endhoven, Nuenen (NL)

(21) Appl. No.: 14/387,976

(22) PCT Filed: Mar. 28, 2013
(86) PCT No.: PCT/IB2013/052498

§ 371 Date: Sep. 25, 2014

Related U.S. Application Data
(60) Provisional application No. 61/617,853, filed on Mar. 30, 2012.

(51) Int. Cl. A61B 17/34

(52) U.S. Cl. CPC A61B 17/3421; A61B 2017/3454; A61B 2017/00991
USPC 604/500; 604/173

(57) ABSTRACT

A nested cannula includes two or more nested tubes (204, 206). A tip portion (202) at a distal end of the nested cannula includes a shape compositely formed from ends of the tubes of the nested cannula to improve one of distal advancement or proximal retraction during deployment.

This disclosure relates to medical instruments and more particularly to nested cannulas having shaped tips to provide a particular function during deployment based on a composite geometry.

Nested cannulas are constructed either based on a patient's 3D image to reach a particular target deep inside the patient, or based on an atlas describing typical anatomy. The nested cannula design is usually created for a specific patient based on a pre-acquired 3D image of a particular anatomical region of the patient, and an identification of a target location within the anatomical region. Specifically, nested cannulas (or a nested cannula configuration) are designed by utilizing the 3D image to generate a series of arc and straight shapes from a particular position and orientation in the 3D image of the anatomical region. The generated arc and straight shapes are utilized to calculate a pathway between an entry location and the target location. The generated pathway is utilized to generate a plurality of nested telescoping tubes that are configured and dimensioned with pre-set curved shapes.

The tubes are typically extended largest to smallest, and a planner specification defines the lengths and the relative orientations between successive tubes to reach the target location. The nested cannula is constructed to stay within permitted regions, and avoid dangerous regions. If the cannula is not advanced correctly, it may cause unwarranted damage.

In accordance with the present principles, a nested cannula includes two or more nested tubes. A tip portion at a distal end of the nested cannula includes a shape compositely formed from ends of the tubes of the nested cannula to improve one of distal advancement or proximal retraction during deployment.

A nested cannula includes two or more nested tubes, each tube having a distal end portion. A composite tip shape extends distally from an end of the nested cannula. The composite tip shape includes the distal end portions of the two or more nested tubes such that when combined the distal end portions collectively contribute to the composite tip shape which improves at least one of distal advancement or proximal refraction during deployment.

A method for employing a nested cannula includes providing a nested cannula having two or more nested tubes, each tube having a distal end portion and a composite tip shape extending distally from an end of the nested cannula, the composite tip shape includes the distal end portions of the two or more nested tubes such that when combined the distal end portions collectively contribute to the composite tip shape, and deploying the nested cannula with the composite tip shape to improve at least one of distal advancement or proximal retraction of the nested cannula.

These and other objects, features and advantages of the present disclosure will become apparent from the following detailed description of illustrative embodiments thereof, which is to be read in connection with the accompanying drawings.

This disclosure will present in detail the following description of preferred embodiments with reference to the following figures wherein:

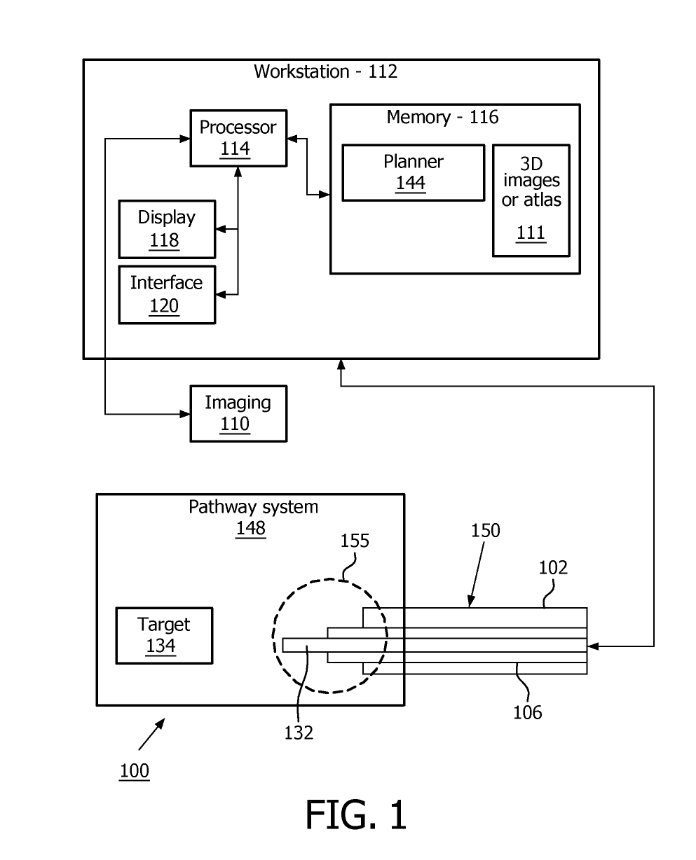

FIG. 1 is a block/flow diagram showing a system for designing and/or employing a nested cannula having a composite tip shape arrangement in accordance with one illustrative embodiment;

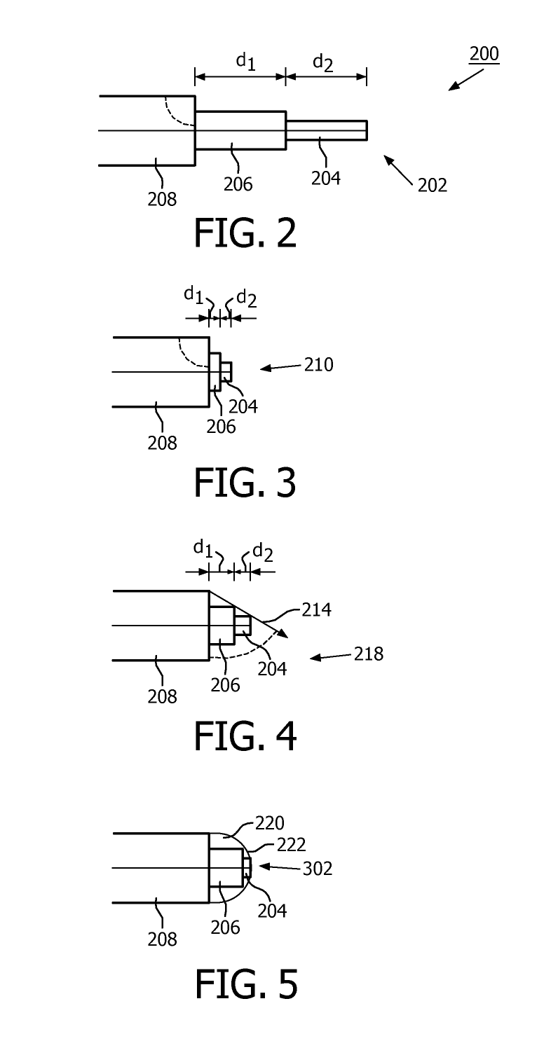

FIG. 2 is a diagram showing a side view of a composite tip of a nested cannula having a stepped arrangement providing a composite shape in accordance with one illustrative embodiment;

FIG. 3 is a diagram showing a side view of a composite tip of a nested cannula having another stepped arrangement in accordance with one illustrative embodiment;

FIG. 4 is a diagram showing a side view of a composite tip of a nested cannula having a stepped arrangement having distances between tubes which follow a relationship in accordance with one illustrative embodiment;

FIG. 5 is a diagram showing a side view of a composite tip of a nested cannula having a rounded relationship of stepped features in accordance with one illustrative embodiment;

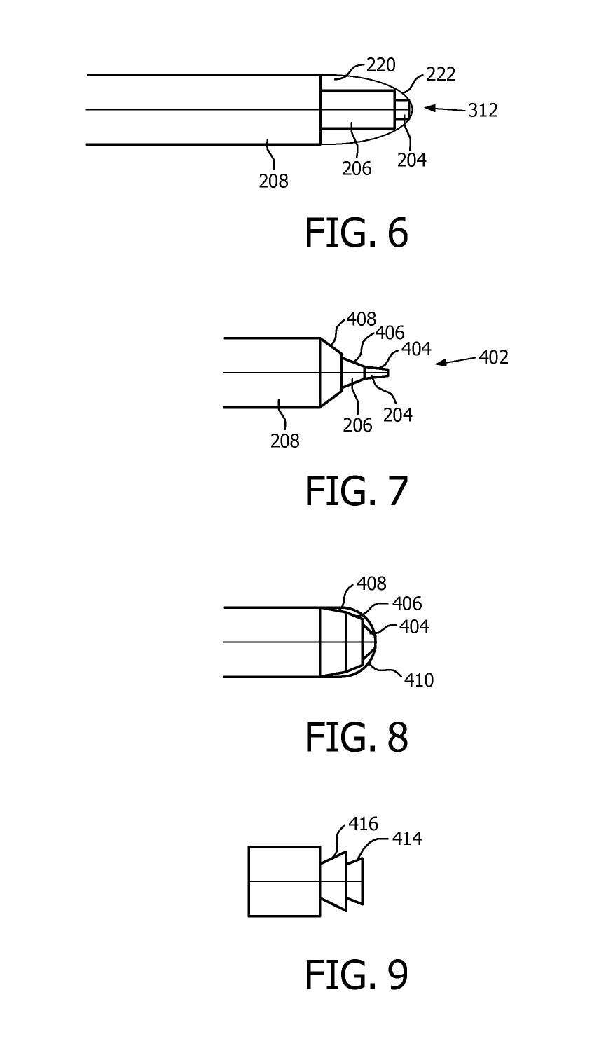

FIG. 6 is a diagram showing a side view of a composite tip of a nested cannula having a parabolic relationship of stepped features in accordance with one illustrative embodiment;

FIG. 7 is a diagram showing a side view of a composite tip of a nested cannula having beveled end portions to create a pointed tip in accordance with one illustrative embodiment;

FIG. 8 is a diagram showing a side view of a composite tip of a nested cannula having beveled end portions to form a substantially rounded tip in accordance with one illustrative embodiment;

FIG. 9 is a diagram showing a side view of a composite tip of a nested cannula having flared end portions in accordance with one illustrative embodiment;

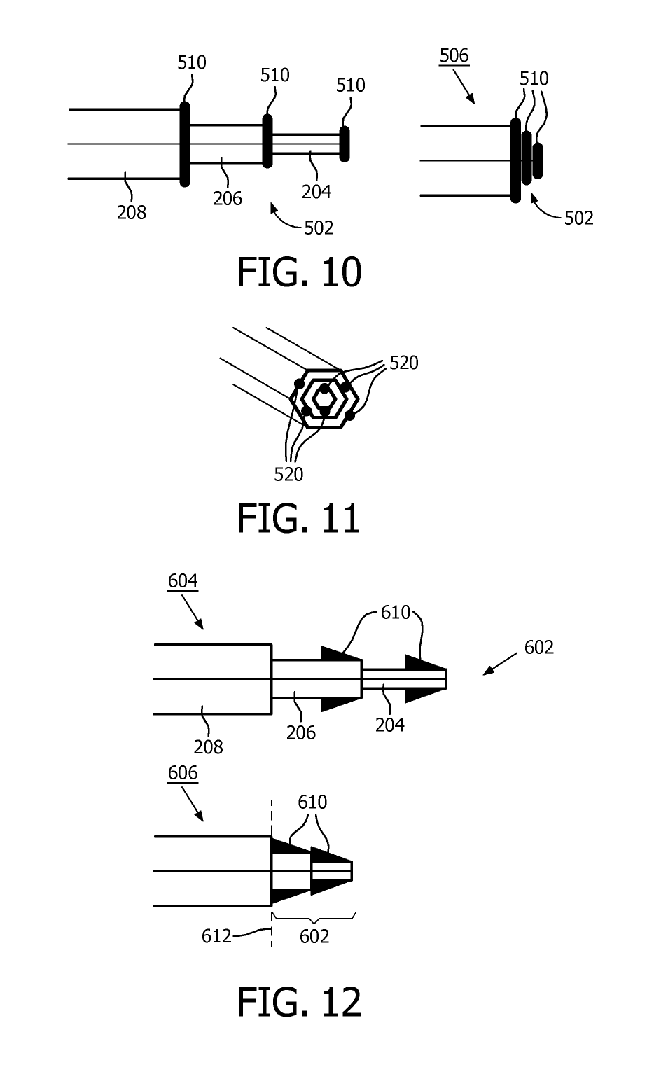

FIG. 10 is a diagram showing side views of a composite tip of a nested cannula being extended and retracted and having beaded end portions in accordance with one illustrative embodiment;

FIG. 11 is a diagram showing a side view of a composite tip of a nested cannula having non-continuous beads in accordance with one illustrative embodiment; and

FIG. 12 is a diagram showing side views of a composite tip of a nested cannula being extended and refracted and having barbed end portions in accordance with one illustrative embodiment.

In accordance with the present principles, systems, devices and methods are provided which include nested cannula arrangements with a tube composite tip that provides a general shape to permit ease of passage of the tip during deployment. A nested cannula device is assembled according to a set of instructions, which indicate the curvature and length of each of a plurality of tubes, any required marks or indicators, and the relative orientation of each tube with respect to a previous tube. Nested cannulas are deployed by extending tubes from a largest to a smallest, that is, they have one degree of freedom, advancement/retraction. They can be inserted into any modeled or imaged region such as an industrial or anatomical region. Since each tube includes all smaller tubes, a ‘net curvature’ (shape) can be computed. Each exposed section of the nested cannula will have a characteristic shape relative to the prior tubes. In addition, each tube will have an orientation relative to its prior and later tubes in the nested cannula.

While nested cannula tubes most likely travel along a primary axis of a tube or organ, it is desirable to make a tip to minimize potential damage in the case of patient or organ motion. Alternatively, the tip may be customized for advancement, while still providing a sharp, but protected inner device. In one embodiment, for example, a tip is designed with a curved end(s) to minimize friction with tissue or other surface that the tip traverses.

In one particularly useful embodiment, the tip is rounded for safety. The rounded shape is achieve by stepped contributions from different tubes in the nested arrangement and can be achieved by patterning cut edges or beveling. Individual tube tips can be flared, such as with a rounded edge or beads so that the tubes can be retracted without pulling them back too far into an enclosing outer tube. In another embodiment, a barbed tip may be employed to set a location or guidewire terminus. Other configurations are contemplated some of which will be illustratively described herein.

It should be understood that the present invention will be described in terms of medical instruments; however, the teachings of the present invention are much broader and are applicable to any industrial instruments. In some embodiments, the present principles are employed in traversing or analyzing complex biological or mechanical systems. In particular, the present principles are applicable to internal access procedures of biological systems, procedures in all areas of the body such as the lungs, heart, brain, gastro-intestinal tract, excretory organs, blood vessels, etc. The elements depicted in the FIGS. may be implemented in various combinations of hardware and software and provide functions which may be combined in a single element or multiple elements.

The functions of the various elements shown in the FIG. 1 can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk—read only memory (CD-ROM), compact disk—read/write (CD-R/W), Blu-Ray™ and DVD.

Referring now to the drawings in which like numerals represent the same or similar elements and initially to FIG. 1, a system 100 which employs nested cannulas in accordance with the present principles is illustratively shown. System 100 may include a workstation or console 112 from which a procedure is supervised and managed. Workstation 112 preferably includes one or more processors 114 and memory 116 for storing programs and applications. Memory 116 may store modules or software tools configured to interpret feedback signals or provide guidance and control of tools employed during a procedure. A planner 144 may be employed to design an instrument 150, such as a nested cannula system or a guide system, by providing arcs, lengths and orientations of cannula segments of the instrument 150 in a patient or pathway system 148. It should be understood that the nested cannula 150 may be deployed manually with or without the use of an imaging 110 system or the console 112.

The instrument 150 illustratively includes three nested cannulas, which are depicted for simplicity and include an outer cannula 102, an inner cannula 132, and an intermediate cannula 106. Two or more nested cannulas are contemplated. The outer cannula 102, inner cannula 132 and intermediate cannula 106 (collectively referred to as the instrument 150) are preferably part of a nested cannula arrangement having a plurality of nested tubes of sequentially varying base dimension (e.g., diameter or thickness) size. In addition, the instrument 150 includes a composite tip shape 155 that provides an advantageous shape for deploying or retracting the instrument 150 in a pathway system 148. Examples of the composite tip shape will be described in greater detail with reference to FIGS. 2-12. Composite refers to the end portion shape where each of the nested tube ends contributes a portion to an overall beneficial shape. In one example, the composite tip shape 155 includes a converging (toward the tip) shape, although other configurations are also contemplated, to permit advancement of the instrument 150 while minimizing friction and reducing the chance of getting hung up on tissue or transitions in a deployment path of the nested cannula arrangement 150.

Workstation 112 may include a display 118 for viewing internal images of the subject 148. In an operative environment, an imaging system 110 may be provided and may include, e.g., a magnetic resonance imaging (MRI) system, a fluoroscopy system, a computed tomography (CT) system, ultrasound (US), etc. Display 118 may also permit a user to interact with the workstation 112 and its components and functions. This is further facilitated by an interface 120 which may include a keyboard, mouse, a joystick or any other peripheral or control to permit user interaction with the workstation 112.

Imaging system 110 may be provided for collecting pre-operative imaging data or real-time intra-operative imaging data. The pre-operative imaging may be performed at another facility, location, etc. in advance of any procedure. These images 111 may be stored in memory 116, and may include pre-operative 3D image volumes of a patient or pathway system 148. Images 111 are preferably employed in designing the instrument 150, e.g., determining its dimensions and orientations for each nested portion for surgery and/or its deployment. Images 111 may also be employed for tracking the instrument 150.

In a particularly useful embodiment, instrument 150 is employed to remove, examine, treat, etc. a target 134 or deliver a tool or substance to the target 134. The target 134 may include a lesion, tumor, injury site, object, etc. During a procedure, the instrument 150 is deployed to reach the target 134. The planner 144 employs the data available for a specific patient's anatomy or an atlas to plan the procedure and design the instrument 150, etc., which is to be proportioned with the other nested components so that it reaches the intended target 134 upon deployment.

Referring to FIG. 2, a nested cannula arrangement 200 shows a stepped composite tip 202 in accordance with one illustrative embodiment. The nested cannula arrangement 200 includes three tubes 204, 206 and 208 in this example. The tubes 204, 206 and 208 are arranged from an outer, largest tube 208 to an inner, smallest tube 204. An intermediary tube 206 is also shown. In this arrangement 200, the tubes 204-208 have a perpendicular cut with respect to the longitudinal axis of the arrangement 200. The tubes 204-208 are extended different distances d₁, d₂, etc. from one another to collectively form the stepped composite tip 202 which provides a pointed shape.

Referring to FIG. 3, in another embodiment, the distances d₁, d₂, etc., can be altered by the design to provide a less pointed and more rounded composite tip 210. By distancing the tubes 204-208 only slightly from one another, a somewhat rounded tip can be achieved. The pattern can be simple, with a constant, but small, added distance (e.g., d₁) for each smaller tube. A net length (e.g., d₁ or other different distances) can be added to the planner (144) to generate the stepped tip 210 (or 202). Note the distances are provided in the design of the instrument by the planner (144) to ensure proper manufacture in accordance with the present principles.

Referring to FIG. 4, in another embodiment, the distance between tubes 204-208 may vary according to a relationship. For example, the arrangement in FIG. 4 includes distances d₁ and d₂ that vary linearly. It should be understood that other relationships may be employed. For example, the distance pattern can vary as a function of the change in outside diameters (or other base dimension), causing a straight line 214 along the corners of the tubes 204-208. The relationship may take many forms, e.g., it may be exponential, parabolic, etc. A composite tip 218 again essentially forms a cone or conic shape, but alternately may take on other stepped shapes depending on the basic shape of the tubes 204-208 (e.g., a pyramidal shape may result from tubes having a square cross-section, etc.). The angle may vary for different applications, taking care that the tip is not so long that it causes navigation errors or issues (e.g., shorter tips are preferable).

Referring to FIG. 5, a composite tip 302 may be arranged to form a continuous rounded pattern. In this embodiment, each tube (e.g., 204-208) includes additional portions 220, 222, which may be formed from the tube material itself, e.g., by machining, molding or otherwise forming the additional portions 220, 222 on the end portion of each tube 204, 206, 208. Additional portions 220 may be attached to tube 208 or tube 206. In this embodiment, composite tip 302 forms a curved end portion that forms a semi-circular shape. Other shapes may also be employed such as, for example, a parabolic shape composite tip 312 as illustratively depicted in FIG. 6. Other designs are contemplated to make the composite tip smoother, sharper, etc., as needed.

Referring to FIG. 7, another embodiment includes a composite tip 402 having beveled edges 404, 406 and 408 on each of tubes 204, 206 and 208. The beveled edges 404, 406 and 408 can help smooth a transition from one tube to the next. In one embodiment, the beveled edges 404, 406 and 408 may be evenly spaced, but may include different bevel angles (FIG. 7). In another embodiment, the beveled edges 404, 406 and 408 are configured to approximate an arc 410 (FIG. 8) or other shape.

Another issue in employing nested cannulas is the ability to retract the tubes without retracting beyond an end of an enclosing tube. Some illustrative examples will now be described that can address this issue.

Referring to FIG. 9, in the illustrative embodiment shown, a nested cannula 420 includes internal tubes having flared end portions 414 and 416. In this case, flared end portions 414 and 416 include a flared end, which is larger than its enclosing tube. In this way, an interior tube cannot pass into its enclosed tube in a proximal direction, e.g., the flared end portions 414 and 416 act as a retraction stop. The flared end portions 414 and 416 may be employed in certain procedures where their flared end provides a beneficial use, e.g., for cell sampling where the objective is to scrape sample cells from a wall.

Referring to FIG. 10, two positions 504 and 506 are depicted for a composite tip 502 in accordance with another embodiment. In this embodiment, beaded end portions 510 are provided on all or some of the tubes. In position 504, the tubes 204, 206, 208 are extended. When retracted in position 506, the beaded end portions 510 act as a retraction stop to prevent further retraction of each tube into its enclosing tube. The beaded end portions 510 include a rounded bead or flanges along the end rim of each tube. Although the largest tube illustrates a bead around it, this is optional and may be employed if there are larger tubes in the series. The beaded end portions 510 also promote ease of deployment as the beads provide less friction and are less likely to hang up on tissue or transitions when being deployed.

Referring to FIG. 11, another embodiment is illustratively shown. In FIG. 10, each of the tubes 204-208 included a full bead around the edge. In FIG. 11, instead of having a full bead about its circumference, beads 520 may be formed or provided on one or more areas to create an enlarged edge. Although the largest tube illustrates a bead (520) on it, this is optional and preferred if there are larger tubes in the series. The beads 520 act as a retraction stop to prevent further retraction of each tube into its enclosing tube.

Referring to FIG. 12, two positions 604 and 606 are depicted for a composite tip 602 in accordance with another embodiment. In this embodiment, barbed end portions 610 are provided on all or some of the tubes. The barbed end portions 610 may include individually spaced apart barbs, a continuous annular barb at each tube or combinations thereof. In position 604, the tubes 204, 206, 208 are extended. When refracted in position 606, the barbed end portions 610 act as a retraction stop to prevent further retraction of each tube into its enclosing tube. The barbed end portions 610 may collectively form a composite conical shape to promote ease of deployment. In other embodiments, a nested cannula tip may be constructed with different tube ends for one or more tubes. For example, a tip may include a single bead on each larger tube ending with a barb on the smallest tube. The composite tip in accordance with the present principles may include any combination of different tube ends. These tube ends may provide different functions and result in different composite shape combinations.

In the case of FIG. 12, an end-point computation may assume that an end of a tube is located at a proximal location shown as a dashed line 612 of a nearest fin of an internal tube. Any distance beyond, the line 612 may be considered the composite tip 602. This type of barbed end portion 610 may be suited when it is desirable to have the tip anchored, perhaps permanently, such as if the inner-most tube 204 is employed as a guide wire to lock into tissue or other substance. The parent tubes (e.g., 206, 208) can then be removed and the inner-most tube 204 can be employed to lead other tools back to the target by traveling over or within the inner-most tube 204.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for nested cannula tips (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws,

What is claimed and desired protected by Letters Patent is set forth in the appended claims:

1. A nested cannula, comprising:
two or more nested tubes, each tube having a distal end portion; and
a composite tip shape extending distally from an end of the nested cannula, the composite tip shape includes the distal end portions of the two or more nested tubes such that when combined the distal end portions collectively contribute to the composite tip shape which improves one of distal advancement or proximal retraction during deployment;
wherein the composite tip shape includes at least one additional portion (220) to form a continuous rounded end portion.

2. The nested cannula as recited in claim 1, wherein the shape compositely formed from ends of tubes comprises a semi-circular shape or a parabolic shape.

3-12. (canceled)

13. A nested cannula, comprising:
two or more nested tubes, each tube having a distal end portion; and
a composite tip shape extending distally from an end of the nested cannula, the composite tip shape includes the distal end portions of the two or more nested tubes such that when combined the distal end portions collectively contribute to the composite tip shape which improves at least one of distal advancement or proximal refraction during deployment;
wherein one or more nested tubes includes a retraction stop on a distal end of the tube which prevents further retraction of a tube into an enclosing tube.

14. The nested cannula as recited in claim 13, wherein the composite tip shape includes a stepped arrangement that forms a conic shape.

15. The nested cannula as recited in claim 14, wherein the stepped arrangement includes different distances between ends of the tubes.

16. (canceled)

17. The nested cannula as recited in claim 13, wherein the composite tip shape includes at least one additional portion to form a continuous rounded end portion.

18. The nested cannula as recited in claim 13, wherein the composite tip shape includes beveled portions.

19. The nested cannula as recited in claim 18, wherein the beveled portions form at least one of a rounded end portion or a conical end portion.

20. The nested cannula as recited in claim 13, wherein the retraction stop comprises flared end portions.

21. The nested cannula as recited in claim 13, wherein the retraction stop comprises beaded end portions.

22. The nested cannula as recited in claim 21, wherein the beaded end portions include a continuous bead about a periphery of interior tubes.

23. The nested cannula as recited in claim 21, wherein the beaded end portions include a non-continuous bead about a periphery of interior tubes.

24. The nested cannula as recited in claim 13, wherein the composite tip shape forms a conical shape and includes barbed end portions, which include barbs to prevent refraction of a tube within its enclosing tube.

25. A method for employing a nested cannula, comprising:
providing a nested cannula having two or more nested tubes, each tube having a distal end portion and a composite tip shape extending distally from an end of the nested cannula, the composite tip shape including the distal end portions of the two or more nested tubes such that, when combined, the distal end portions collectively contribute to the composite tip shape;
wherein the composite tip shape includes at least one additional portion to form a continuous rounded end portion; and
deploying the nested cannula with the composite tip shape to improve at least one of distal advancement or proximal refraction of the nested cannula.

26. A method for employing a nested cannula, comprising:
providing a nested cannula having two or more nested tubes, each tube having a distal end portion and a composite tip shape extending distally from an end of the nested cannula, the composite tip shape including the distal end portions of the two or more nested tubes such that, when combined, the distal end portions collectively contribute to the composite tip shape;
wherein one or more nested tubes includes a refraction stop on a distal end of the tube which prevents further retraction of a tube into its enclosing tube; and
deploying the nested cannula with the composite tip shape to improve at least one of distal advancement or proximal refraction of the nested cannula.
